(19) United States
(12) Patent Application Publication
(10) Pub. No.: US 2025/0380968 A1
(43) Pub. Date: Dec. 18, 2025

(54) METHODS AND APPARATUS FOR JOINT REPAIR

(71) Applicant: WRIGHT MEDICAL TECHNOLOGY, INC., Memphis, TN (US)

(72) Inventors: Murray John PENNER, Vancouver (CA); Shawn E. McGINLEY, Arlington, TN (US); Jesse G. MOORE, Germantown, TN (US); William BRAGG, Lakeland, TN (US)

(73) Assignee: WRIGHT MEDICAL TECHNOLOGY, INC., Memphis, TN (US)

(21) Appl. No.: 19/280,278

(22) Filed: Jul. 25, 2025

Related U.S. Application Data
(63) Continuation of application No. 18/062,071, filed on Dec. 6, 2022, now Pat. No. 12,396,755.
(60) Provisional application No. 63/267,240, filed on Jan. 28, 2022.

(51) Int. Cl. A61B 17/56; A61B 17/16; A61B 17/17; A61B 17/72; A61F 2/30; A61F 2/42; A61F 2/46; A61F 2/66

(52) U.S. Cl. CPC A61B 17/562; A61B 17/1633; A61B 17/1642; A61B 17/1682; A61B 17/1775; A61B 17/1739; A61B 2017/564; A61B 2017/567; A61B 2017/568; A61B 17/7291; A61F 2002/30878; A61F 2002/30879; A61F 2002/30891; A61F 2002/30892; A61F 2/4202; A61F 2002/4205; A61F 2002/4207; A61F 2/4606; A61F 2/6607

(57) ABSTRACT

The disclosed subject matter relates to a system and method for preparing a surface of a bone proximate a joint, in which the preparation includes boring a plurality of arced channels/troughs in the bone surface using an arch drill assembly guided by plural guide bores along a predetermined longitudinal arc. The bored troughs create a scalloped surface on the bone to which an inverse contoured joint insert/implant having cooperating convex ridges engages. The plural guide bores are also defined by the predetermined longitudinal arc which may be circular or helical. The disclosed subject matter minimizes bone removal and allows greater access options for preparing the bone surface.

### CROSS-REFERENCE TO RELATED APPLICATIONS

This application is a continuation filed under 37 C.F.R. § 1.53 claiming the benefit under 35 U.S.C. § 120 of any and all applications for which a foreign or domestic priority claim is identified in the Application Data Sheet as filed with the present application, including U.S. patent application Ser. No. 18/062,071, filed Dec. 6, 2022, which claims priority to U.S. provisional application 63/267,240 entitled “METHODS AND APPARATUS FOR JOINT REPAIR” filed Jan. 28, 2022, and are hereby incorporated by reference in accordance with 37 C.F.R. §§ 1.57; 1.97; and 1.98 in their entireties.

### TECHNICAL FIELD

The disclosed subject matter relates generally to a system and method for preparing a bone surface to accept a joint insert while minimizing bone material removal and creating a secure seat for the joint insert. The disclosed subject matter bores (reams, machines, drills) a plurality of laterally arranged concave channels (troughs) in the joint end of the bone, using an arced drill drive and a drill guide with a plurality of arced guide bores and provides the joint insert with cooperating convex ridges, such that the joint insert will positively seat with minimal need of pins, pegs or screws to provide stability.

### BACKGROUND

Removing minimal amounts of talar and tibial bone is especially challenging in osteopenia patients. Thus there is a need for more precise preparation with a minimal depth of bone removal. Current anterior TAR techniques take a large amount of bone from a flat cut in a concave surface. Other require a lateral approach which has other disadvantages. The anterior and lateral approaches have pros and cons regarding their respective soft tissue complications, osteotomy necessity, orientation of the bone cut and gutter visualization. FIG. 1 illustrates a prior art technique showing the tibia 102, talus 103 and the fibula 105. As shown through the progression in FIG. 1, the tibia 102, and talus 103 are both shaped to have flat surfaces 112 and 113 respectively, because of the concave shaft of the joint end of the tibia 102, a significant amount of heathy bone is removed. An improved anterior TAR technique and instruments are needed.

One prior art method for drilling channels in bone, described in Patent Publication US 2005/0267481, includes attaching a drill assembly to the frame assembly, so a drill bit of the drill assembly will follow predetermined circular path in fixed relation with the frame and moving the drill bit through the predetermined path while the drill bit is rotating to cut the channel via a pivot arm. The prior art method secures the drill assembly including the drill bit to the pivot arm assembly and rotates the drill bit using the pivot arm assembly so the drill bit traverses the predetermined path. However, the apparatus is complicated, not user friendly, limited to a single predetermined path, requiring reconfiguration/relocation of the frame for each desired channel, is incapable of following helical paths and is directed to joining bones.

### SUMMARY

The embodiments described herein are directed to a system and method for bone surface preparation in joint repair. In addition to or instead of the advantages presented herein, persons of ordinary skill in the art would recognize and appreciate other advantages as well.

In some embodiments, a system for repairing a joint is presented. The system includes a joint insert; a drill guide; and, a drill assembly. The joint insert including a scalloped top surface configured to rigidly engage a prepared complimentary bone surface; a bottom surface configured to non-rigidly engage a joint surface, the bottom surface opposing the top surface. The drill guide of the presented system having a plurality of bores defined by a circular cross section and a longitudinal arc, the bores cooperate with the drill assembly to direct the sheath on a single predetermined path defined by the longitudinal arc of the respective bores. The drill assembly of the system including a rigid sheath, a flexible drive shaft within the sheath, a cutting bit at the distal end of the sheath driven via the flexible drive shaft and a driver.

The disclosed subject matter also presents a joint insert having a top surface configured to engage a prepared bone surface proximate to a joint and a bottom surface configured to non-rigidly engage a joint surface, the bottom surface opposing the top surface. The joint insert including a longitudinal axis extending from the front to the rear of the insert and a lateral axis perpendicular to the longitudinal axis; the top surface having a plurality of convex ridges, each convex ridge having an outer surface intersecting the outer surface of laterally adjacent ridges, the outer surface of each ridge defined by a constant radius longitudinal arc and a circular cross section perpendicular to and along the arc. The ridges of the joint insert configured to engage corresponding concave troughs in a prepared bone surface. In another embodiment, the ridges may be smoothed down with a secondary step involving an end shrouded side cutting arced reamer tool to accommodate non-ridged implants, along with a corresponding guide for ridge take-down. The ridges may be retained, reduced, or removed depending on surgeon preference and implant to bone interfacing.

In some embodiments, a drill guide for preparing a bone surface proximate to a joint is presented. The drill guide including a plate with front and opposing rear faces and a plurality of bores through the plate. Each of the plurality of bores are defined by a longitudinal arc and a circular cross section perpendicular to and along the arc and are configured to cooperate with a sheath of a drill assembly having substantially the same respective circular cross section and longitudinal arc, and direct the sheath on a single predetermined path defined by the longitudinal arc of the respective bores.

In yet other embodiments, a method for repairing a joint between two bones is presented. The method includes exposing the joint; boring a plurality of adjacent troughs in the proximal or distal end of one of the bones to form a scalloped surface; and, seating a joint insert with a cooperating surface on one side on the joint insert on the prepared scalloped surface.

### BRIEF DESCRIPTION OF THE DRAWINGS

The features and advantages of the present disclosures will be more fully disclosed in, or rendered obvious by the following detailed descriptions of example embodiments. The detailed descriptions of the example embodiments are to be considered together with the accompanying drawings wherein like numbers refer to like parts and further wherein:

FIG. 1 is an illustration of a prior art method for preparing a bone surface in the ankle;

FIG. 2A is an illustration of a scalloped bone surface associated with embodiment of the disclosed subject matter;

FIG. 2B is an illustration of the boring of troughs in the tibia with a drill assembly to create the scalloped bone surface illustrated in FIG. 2A in accordance with embodiments of the disclosed subject matter;

FIG. 3 illustrates the drill guide positioned on the ankle in accordance with embodiments of the disclosed subject matter;

FIG. 4 illustrates an example of a plurality of bores which define the scalloped surface of the bone forming a saddle according to an embodiment of the disclosed subject matter;

FIGS. 5A and 5B illustrate the bore defined by the drill guide and the resultant scalloped surface in accordance with embodiments of the disclosed subject;

FIGS. 6A-6C illustrate drill guides with keyed guide bores according to embodiments of the disclosed subject matter;

FIG. 7 illustrates a drill guide with keyways in the guide bores according to embodiments of the disclosed subject matter;

FIG. 8 illustrates helical bores defining the scalloped surface according to embodiments of the disclosed subject matter;

FIGS. 9A-9D illustrate various views of a joint insert cooperating with the scalloped bone surface according to embodiments of the disclosed subject matter;

FIG. 10 illustrates a prior art sleeved drill assembly;

FIG. 11 illustrates a method of preparing a scalloped bone surface according to embodiments of the disclosed subject matter;

FIGS. 12A, 12B illustrate cooperating joint surfaces of an insert pair and cross sections thereof according to an embodiment of the disclosed subject matter;

FIGS. 13A and 13B illustrate a sleeved drill assembly and bit respectively for use with embodiments of the disclosed subject matter;

FIGS. 14A and 14B illustrate anterior entry ports for the reamer according to embodiments of the disclosed subject matter;

FIG. 15 is a flow chart describing the operation of boring with the use of robotic guidance according embodiments of the disclosed subject matter; and,

FIG. 16 illustrates a clamshell drill guide according to an embodiment of the disclosed subject matter.

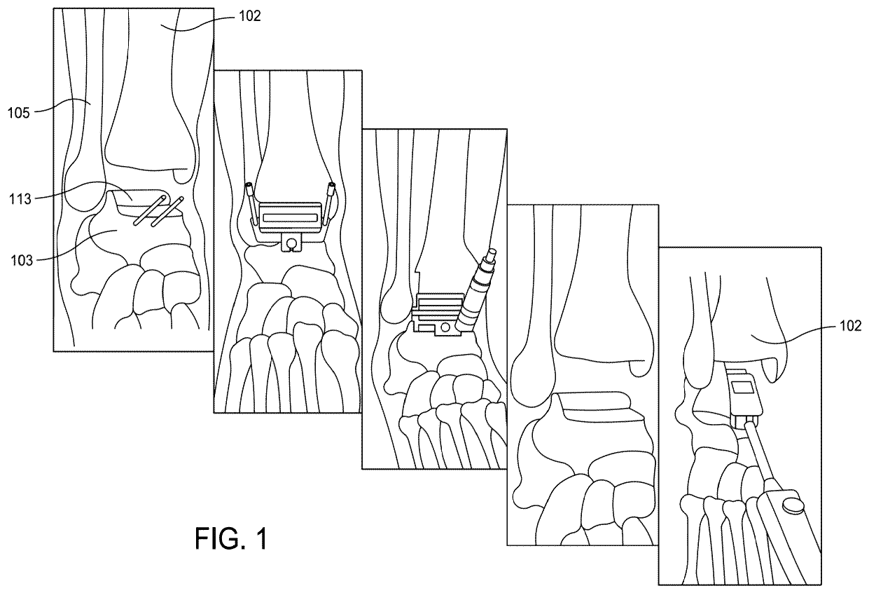

### DETAILED DESCRIPTION

The description of the preferred embodiments is intended to be read in connection with the accompanying drawings, which are to be considered part of the entire written description of these disclosures. While the present disclosure is susceptible to various modifications and alternative forms, specific embodiments are shown by way of example in the drawings and will be described in detail herein. The objectives and advantages of the claimed subject matter will become more apparent from the following detailed description of these exemplary embodiments in connection with the accompanying drawings.

It should be understood, however, that the present disclosure is not intended to be limited to the particular forms disclosed. Rather, the present disclosure covers all modifications, equivalents, and alternatives that fall within the spirit and scope of these exemplary embodiments. The terms “couple,” “coupled,” “operatively coupled,” “operatively connected,” and the like should be broadly understood to refer to connecting devices or components together either mechanically, electrically, wired, wirelessly, or otherwise, such that the connection allows the pertinent devices or components to operate (e.g., communicate) with each other as intended by virtue of that relationship.

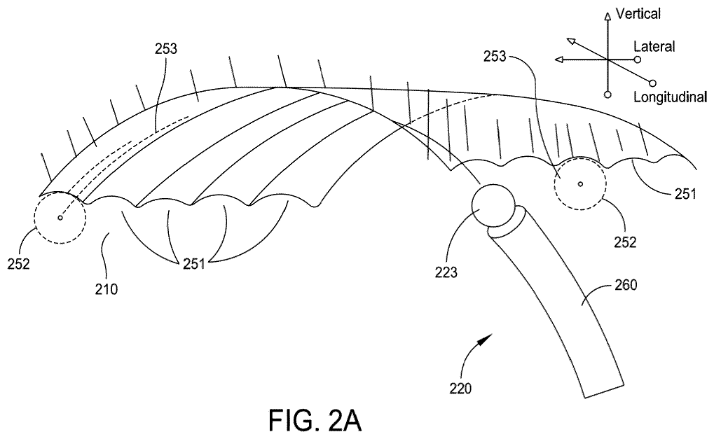

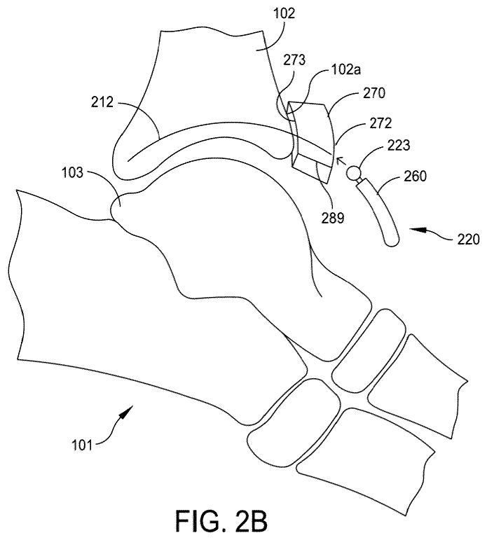

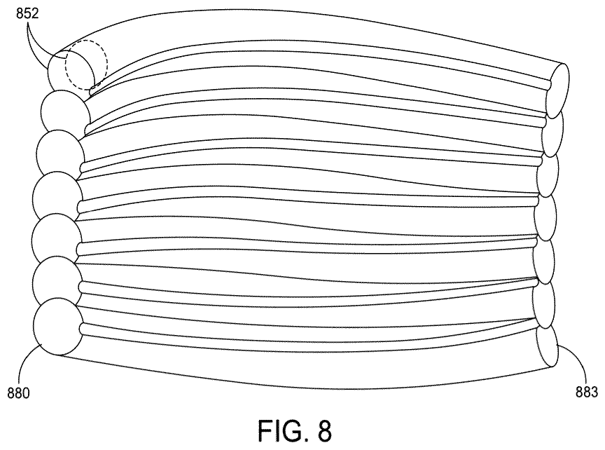

FIG. 2A illustrates an example of a scalloped bone surface 210 of and/or created by embodiments of the disclosed subject matter, the scalloped bone surface 210 includes a plurality of adjacent troughs 251 which are defined by an arc 253 extending generally in the longitudinal direction and a circular cross section 252 in the lateral direction (perpendicular to the arc) along the arc 253 forming the defining bores 280. In forming the scalloped surface 210, the circular cross sections 252 defining each trough 251 intersect the circular cross sections 252 defining adjacent troughs 251. As shown in FIG. 2A, the longitudinal arc 253 is circular, however the arc may also be helical as described in FIG. 8. Helical arcs present additional access advantages in which drill bit 223 may better avoid the tibial anterior tendon or neurovascular bundle, yet still create the desired scalloped surface 210. FIG. 2B shows the arrangement of the drill guide 270 with respect to the tibia 102 and the talus 103 of ankle 101. The rear surface 273 of the drill guide 270 may be advantageously contoured to match the vertical surface 102a of the tibia 102. The drill assembly 220, including the bit 223 and sheath 260 are guided via guide bore(s) 281 in the drill guide 270 to remove bone in order create adjacent troughs at the end of the tibia 102 to form the prepared surface 210 (the prepared tibia surface 212 as shown). The circular cross section 252 of the bores for example when used for the tibia and/or talus may be around 6 mm in diameter.

The arched bores 280 may be reamed (bored/drilled) in parallel planes. Additionally, troughs 251 can be reamed in planes angularly offset from one another with same or different arc diameters to create the scalloped shape of the prepared bone surface 210. The drill guide and implant surfaces may correspond (cooperating) in contours for any geometry (topography) and predetermined sizes. The center of rotation for the arcs may extend inward, parallel, or outward. Additionally, the guide bores 281 of the drill guide 270 directs the drill assembly 220 in a repeatable position to ensure contours (troughs) match the joint implant contours (convex ridges). The drill guide may further include docking points, with two lateral tabs with a through hole for pinning so as to retain the relative position between bone and drill guide. This system may also lock/interface with surgical pins or frames already in place and there are many variations on the design of a drill assembly 220 to work around the anatomy while providing positive registration of the guide(s) and drill assembly 220. Registration may include orientation steps to coordinate patient anatomy with a guided instruments or computer system using reference bodies, optical, haptic, or fluoroscopic markers to orient surgical steps in 3D space with the patient anatomy. Additionally it is envisioned that arthroscopic techniques for lavage of debris and thermal regulation of the surgical site may be utilized in embodiments of the disclosed subject matter.

While not shown, both the tibia surface and talus surface may be prepared using the same drill guide 270 and drill assembly 220 by repositioning the drill guide 270 from the tibia to address the talus. Alternatively, the bores 280 may be sized such that both the tibia and talus are prepared simultaneously by the advancement of the drill bit 223 along the defined arc, cutting the scalloped surface of the tibia 212 with the top of the drill bit 223 and cutting the scalloped surface (not shown) of the talus 103 with the bottom of the drill bit 223 at the same time.

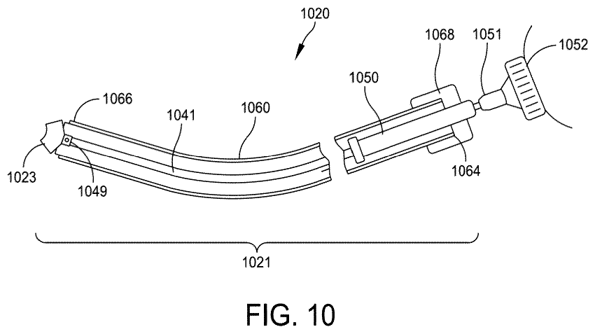

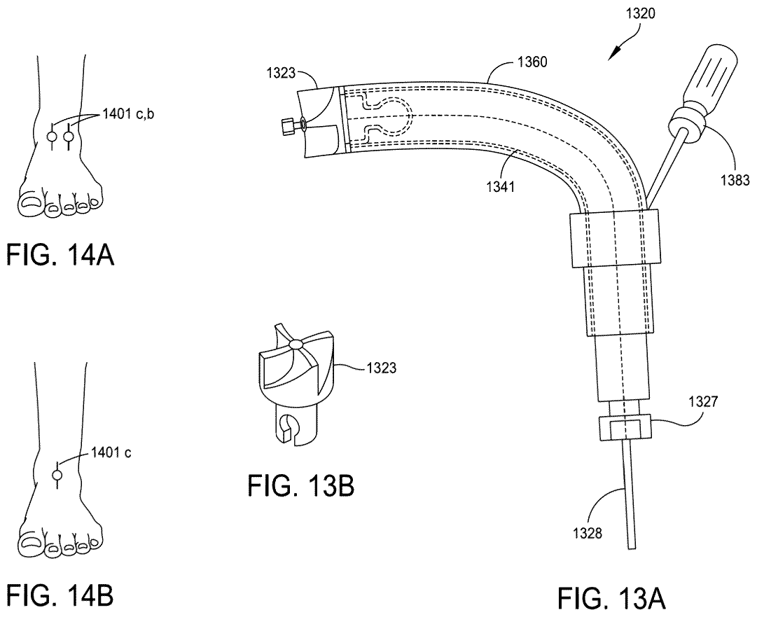

The cutting drill bit 223 may extend along the path while the drill assembly is held by hand, or a robotic controlled arm, restricted by the drill guide 270. The drive portion of drill assembly 220 need not be aligned with the predetermined path but may be introduced utilizing a 90 degree gear box to convert torque to the arc sleeve/sheath 260 of the drill assembly 220. The drill assembly 220 may utilize flexible structures known in the art (e.g. laser cut modular head IM reamer). Alternative embodiments may utilize an in-line power driver, such as U.S. Pat. No. 5,395,188 discussed below with respect to FIG. 10, along with modifications to enable its use as surgical equipment or the embodiment described with respect to FIGS. 13A and 13B. FIG. 13A shows the drilling assembly 1320 sans the driver. The drilling assembly 1320 includes a rotating bit 1323, a sheath 1350 and flexible drive shaft 1341. Additionally, an anti-torque device/handle/robot fixture 1383 may advantageously hold the drilling assembly 1320 and prevent the sheath from rotating with the bit 1323. FIB. 13B is an isometric view of the rotating bit 1323 according to some embodiments of the disclosed subject matter. A guide wire 1328 may be used for directing the bit to the entry point of the bore, the guide wire 1328 runs through the center of the driver attachment 1327, sheath 1360, drive shaft 1341, and through the center of the bit 1223.

An example of a drill assembly is described in U.S. Pat. No. 5,395,188, the entirety of which is hereby incorporated by reference. Turning to FIG. 10, for guiding the drill bit 1021 along the curved path to drill a hole in a desired direction, the drill assembly 1020 includes a curved guide sleeve or tube 1060 between an inlet end 1064 and terminating in an outlet end 1066. For supporting the drill bit 1021 in the guide tube 1060, a cap bushing 1068 is threaded securely to the inlet end 1064 of the guide tube 1060. The bushing journals the drill shaft 1050 for rotating and sliding movement through the curved tube thereby enabling the drill bit 1021 to project outwardly through the outlet end 1066 of the tube 1060 into an adjacent surface through which a hole is to be drilled. The flexible shaft 1041 of the drill bit assembly 1021 is a stiffly-flexible coiled wire cable, the coils of which are tightly wound to produce a strong flexible shaft. The cutting bit 1023 is secured to one end of the cable 1041 by a sleeve connector 1049 crimped and secured to the cutting bit 1023 and receiving an inserted end of the cable. For engagement with a power drill tool 1052, the opposite end of the flexible cable 1041 is inserted in a stainless steel sleeve 1050 and secured thereto by a bushing or collar 1051.

With the use of the drill guide 270, the drill bit 223 of the drill assembly 220 follows a predetermined path, more particular a predetermined circular path, or predetermined helical path, in fixed relation with respect to the drill guide 270 while moving the drill bit 223 through the predetermined path while the drill bit 223 is rotating, so as to cut the channel/trough 251. The nature of the guide bores 281 within the guide 270 each correspond to one particular circular or helical path (including position and orientation of the path), such that the drill assembly (i.e., sleeve/sheath 260 and bit 223) cooperates with the guide 270 to follow substantially the path defined by the guide bore 281.

Alternatively, rather than a drilling/boring assembly that utilizes a traditional bit (i.e. rotating cutting head), the channels/troughs 251 may be formed using an ultrasonic or laser cutting heads. An ultrasonic head uses ultrasonic waves to fragment, emulsify and aspirate soft tissue and bone. One example of the use of an ultrasonic head is Stryker's Sonopet® ultrasonic Aspirator. The use of a laser head for the ablation of bone tissue allows higher accuracy and increases bone healing compared to conventional mechanical bone cutting. Both these boring heads also allow for boring channels/troughs have non-circular cross sections, as they do not rely on a rotating bit. For example, elliptical, parabolic, V-shaped, U-shaped or even irregular troughs may be bored with these alternative heads. Additionally, with precision guidance tapered troughs may be implemented with the use of these heads.

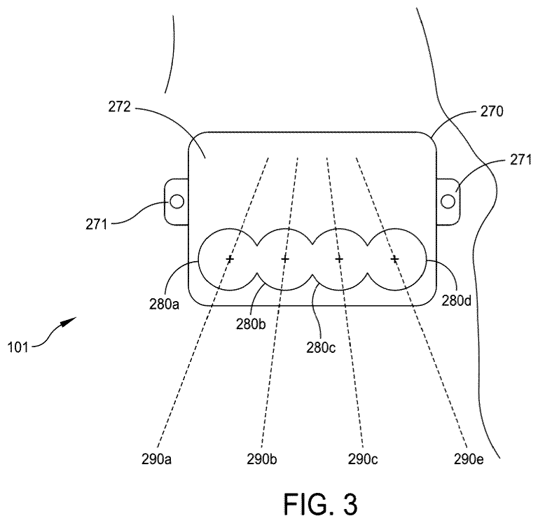

FIG. 3 illustrates a front view of the drill guide 270 on the ankle 101. The front face 272 of the drill guide 270 has a plurality of guide bores 281a-d that are configured to guide the drill assembly 220. While a plurality of guide bores are shown, single guide bores allowing a plurality of reaming sweeps are likewise envisioned. FIGS. 14A and 14B illustrate multiple entry portals 1401a and 1401b and a single portals 1401c respectively each resulting in multiple sweeps to form the prepared surface of the disclosed subject matter. The entry portals typically correspond to the number of guide bores, however each portal may be expanded to received multiple sweeps. The drill guide 270 may include attachment points 271 that allow the rigid attachment of the drill guide 270 to the bone, in this case the tibia 102. Each of the guide bores 280a-d are defined as noted above by a circular cross section 252 along an arc 253 or helix. As shown in FIG. 3 each of the arcs 253 may lie within respective planes 290a-d that are not parallel to one another. The defining planes 290a-d in which the defining arc 253 lie may be parallel or oblique and their intersections may be located outside of the guide 270, with respect to helical arcs, such a plane is not defined.

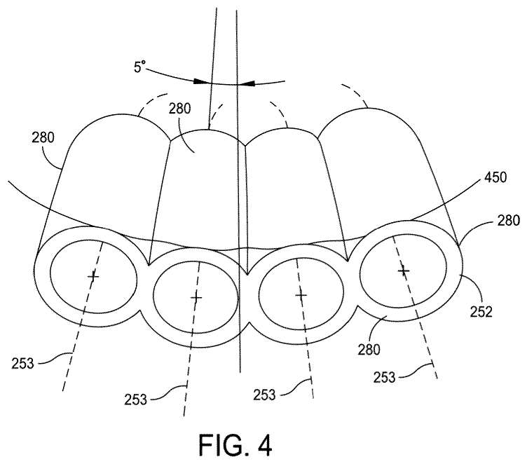

FIG. 4 illustrates a plurality of bores 280 which define the scalloped surface 210 of the bone formed in a saddle. As shown in FIG. 4, a lateral curve 450 tangent to each of the circular cross sections 252 of the respective bores 280 has a positive curvature (meaning the center of curvature is above) and thus will form a convex surface on the bone surface, while the arcs 253 or helix defining the bores 280 have a negative curvature (meaning the center of curvature is below) and thus will form a concave surface on the bone surface. These convex curvatures in the lateral direction along with the concave curvature in the longitudinal direction will result in a saddle topography for the scalloped surface. It is also envisioned the scalloped surface 210 may be formed with a saddle in the opposite direction, such that the scalloped surface is concave in the lateral direction and convex in the longitudinal direction. Moreover, the scalloped surface 210 may be defined as semi-hemispherical in that both in the lateral and longitudinal directions, the surface curves are both positive or both negative.

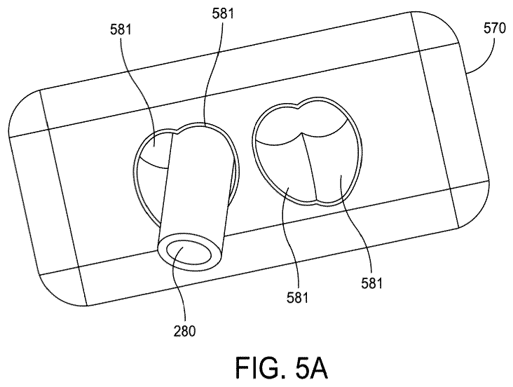

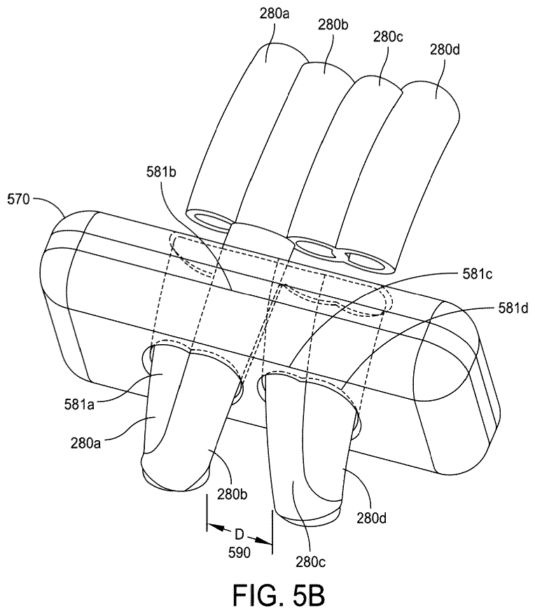

In FIG. 5A, the drill guide 570 is shown with four guide bores 581a-d, the first set 581a, 581b intersecting within the guide 570 and the second set 581c, 581d also intersecting within the drill guide 570. The orientation of the guide bores 581 within drill guide 570 allow for off center access to create the scalloped surface. As noted earlier, this access advantageously avoids the tibialis anterior tendon and/or neurovascular bundle. FIG. 5B further illustrates this access in which the adjacent bores 280 overlap to create the desired scalloped surface, yet the bores 280 are separated laterally a distance D 590 as they approach the drill guide 570. This approach for example may allow a 10 degree medial entry, as well as lateral entry. Entry into the joint space may include arthroscopic techniques known in the surgical arts. In one embodiment, the arched reamers may pass through a single portal to create an array of contoured sweeps to remove bone. In another embodiment, there may be multiple portals which allow for sweeps at different entry angles to form a singular prepared joint surface within the patient's anatomy. In the case of multiple incision portals, the implant may comprise multiple components which are partitioned outside of the patient and assembled within the patient to combine and operate as an implant system.

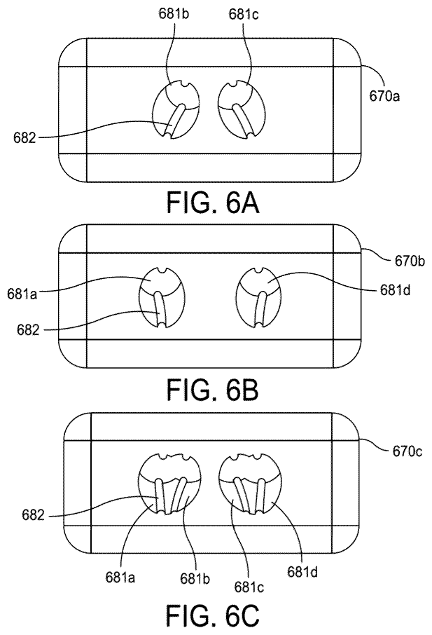

FIG. 6A shows a drill guide 670a with two guide bores 681a and 681b. The bores defining the guide bores 681b,c are inclined towards each other intersecting behind the drill guide 670a. The guide bores 681b and 681c also include a key 682 at the top and bottom of the guide bores 681, while two are shown for each guide bore, multiple keys, a single key or no keys are envisioned. The key 682 interacts with a respective keyway on the drill assembly 220 to precisely guide the drill assembly 220 along the predetermined arc defining the guide bores 681 and desired scalloped surface. FIG. 6B shows guide bores 681a and 681d in drill guide 670b these guide bores diverge from one another and thus the defining arcs intersect in front of the drill guide 670b. Guide bores 681a and 681d also include keys 682 that interact with respective keyways on the drill assembly (not shown). Drill guides 670a and 670b may be used sequentially to prepare the scalloped surface, boring the troughs associated with guide bores 681b and 681c, then positioning drill guide 670b and boring the troughs associated with guide bores 681a and 681d. The same guide bores 681 may also be incorporated into one drill guide 670c as shown in FIG. 6C. In FIG. 6C, the set of guide bores 681a-d are created in drill guide 670c. While the guide bores overlap, the shape of the bores and the respective keys define four distinct arc paths and thus result in the same scalloped surface as if both the drill guides 670a and 670b were uses sequentially, however advantageously, the use of the combined drill guide 670c only requires one positioning of the drill guide, reducing time, effort and opportunities for misalignment.

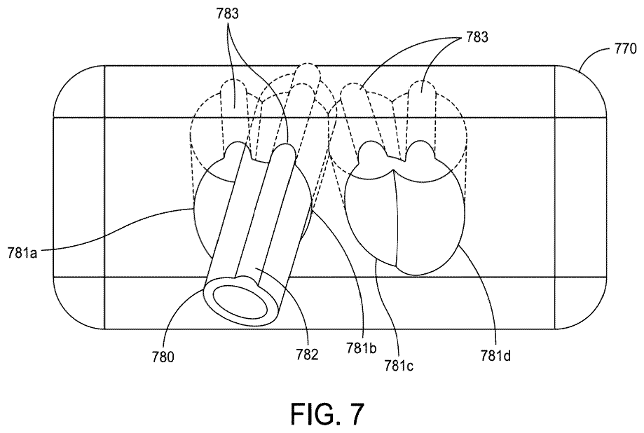

FIG. 7 illustrates a drill guide 770 in which the keyways 783 are defined within the guide bores 781a-d and the key 782 is on the sleeve/sheath of the drill assembly defined by bore 780. In the drill guides 770 utilizing keys or keyways, the configuration of the drill bit 223 with respect to the drill assembly sleeve 260 may require adjustments. For example in the drill guide 770 with keyways in the guide bores 781, the drill bit may be of a larger diameter than the guide bores to allow the key on the drill assembly to follow the bit along the predefined arc into the bone, resulting in the subsequently created troughs of the scalloped surface to have the same radius as the bit rather than matching the sleeve of the drill assembly. Alternatively, the key 782 on the sheath may not extend along that portion of the sheath that extends into the bone, or may be located on the opposite side of the bore than that side which creates the troughs. For example, if the bottom surface of the tibia is to be prepared the key on the sleeve would be on the bottom of the bore since only the upper portion of the bore defines the troughs 251, whereas, if the top of the talus bone is being prepared via the bore, the key on the sleeve would be positioned on the top portion of the bore as to avoid contact with the talus as the drill assembly advances into the bone. Similarly, where the key is fashioned within the guide bores as shown in FIGS. 6A-D, the drill bit 223 may need to be attached to the drill assembly from the rear of the drill guide 670, such that it would not interfere with the key 682 of the drill guide 670, it such cases in may be advantageous to further recess the rear surface of drill guide to countersink the bit so that the rear surface still matches the contour of the bone. As may be appreciated, the scalloped surfaces may be prepared with the use of a single drill guide, a single drill guide repositioned one or more times or multiple drill guides used sequentially. The drill guides may be patient specific instruments designed to match the contour of the patient bone and align implantation cuts with a pre-planned position. The guides may also be selectable from a selection of standardized guides.

FIG. 8 illustrates the bores that define a scalloped surface in which the arcs are helical. The circular cross sections 852 are along the helical arcs. The bores 880 define both the troughs of the scalloped bone surface but also the guide bore (not shown) and the sheath of the drill assembly, in this case a keyway 883 is also shown. It should be noted that throughout the disclosure examples are presented in which each of the bores that define the guide bores, the troughs of the scalloped surface, and the ridges of the inserts have been shown with the same diameter circular cross section, however adjacent bores need not have the diameter and in some circumstances different diameter cross sections may be advantageous for several reasons including to minimize bone removal and/or stress concentrations. The arches defining the troughs and insert ridges may be in helical form as shown in FIG. 8, may be formed tight, wide, or straight (no longitudinal curvature), additionally as described elsewhere, the respective adjacent troughs may have various orientations with respect to each other, e.g. parallel, oblique, clam shell, overlapping, intersecting etc. It is envisioned that the arches may have radii ranging from 0 (a straight bore) to a 100 mm, with a bore diameter of 3-20 mm. The arches need not have a round cross section in the case of ultrasonic cutting tips or laser heads. The desired bone boring shape may utilize one tool path or a combination of straight and arched tools to achieve a net shape cavity in the patient bone.

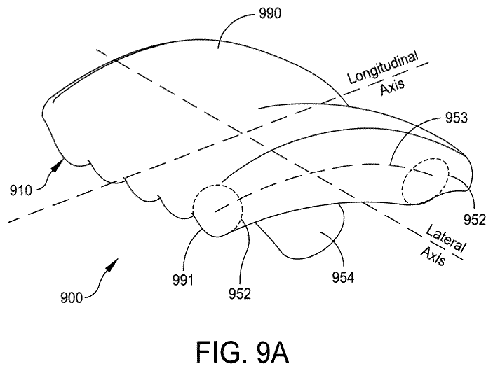

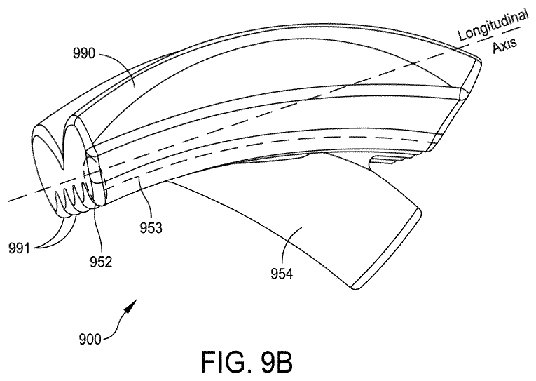

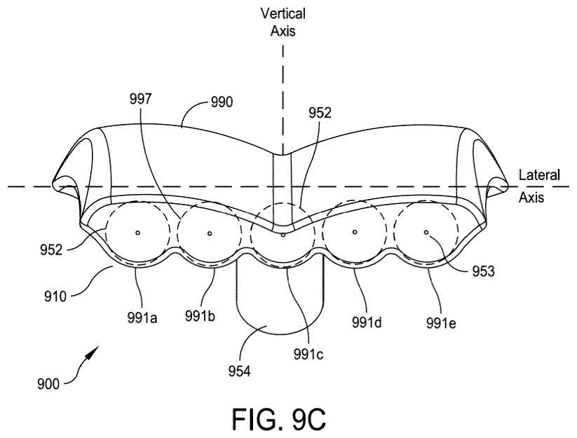

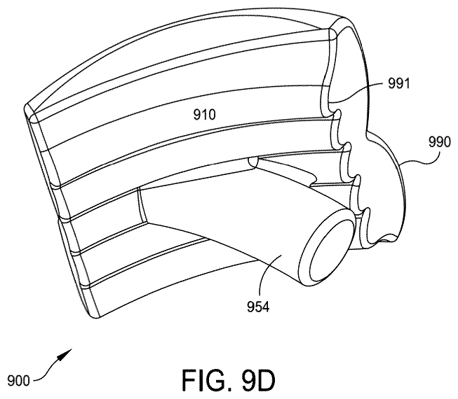

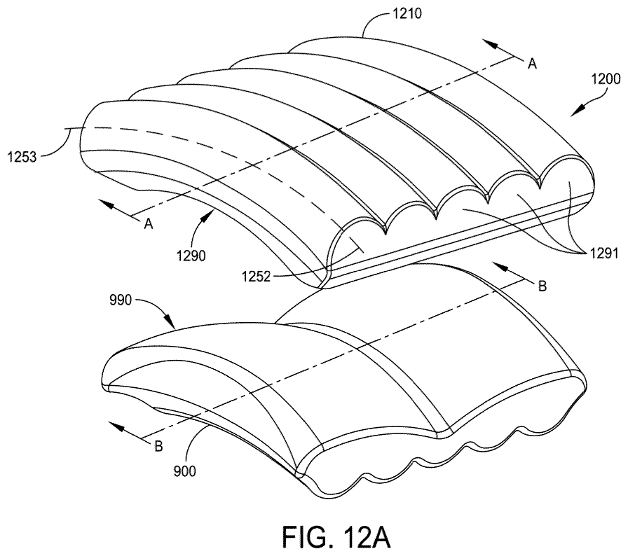

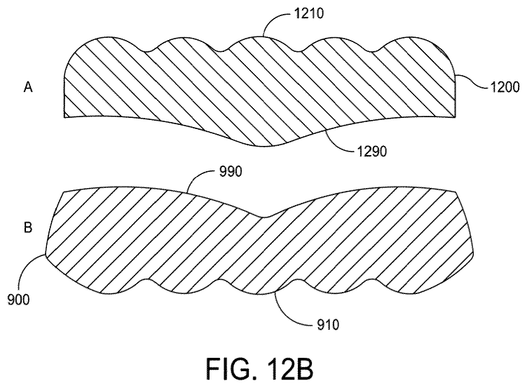

An example of a joint insert (implant) 900 for engaging the prepared scalloped bone surface described above is shown in FIGS. 9A-9D. The insert 900 has a joint surface 990 proximate the joint and flexibly engages an opposing joint surface (as shown in FIG. 12A). The insert 900 has a bone engaging surface 910 that is shaped as the inverse of the scalloped bone surface discussed previously. Specifically, the bone engaging surface 910 includes a plurality of convex ridges 991, each of the convex ridges 991 defined by a circular cross section 952 along and arc 953, these convex ridges cooperate with the troughs of the scalloped bone surface defined with substantially similar circular cross sections 252 and arcs 253. Each of the convex ridges 991 extend along the longitudinal axis and are laterally adjacent to the other convex ridges 991 across the engaging surface 910. As the bone engaging surface 910 of the joint insert 900 is an inverse of its corresponding scalloped bone surface, its surface likewise may be formed with a saddle topography, be convex in both the lateral and longitudinal axis, or be concave in both the lateral and longitudinal axis. Other characteristic topographies that may be defined by the arrangement of adjacent arced bores are also envisioned.

Also shown in FIGS. 9A-9D, a peg 954 may be received into a cooperating peg bore (not shown) in the scalloped bone surface 210 to aid in rigidly attaching the insert 900 to the bone surface. The peg 954 may be integral to the joint insert 900, or may be inserted subsequently through an aperture (not shown) in the insert 900. Additional pins, pegs and screws may also be used to rigidly attach the insert to the bone, however given the minimization of sliding paths due to the interaction of the convex ridges 991 of the insert 900 and concave troughs 251 of the scalloped bone surface 210, such additional attachment elements may not be necessary. Cavities for pegs may be formed with arched reaming drills and guides in a similar manner as the bone removal, but with inverted arch direction (see implant FIG. TBD). The joint insert 900 may be machined, molded, casts forged or 3D printed and may be constructed of ceramic, Ultra-high-molecular-weight polyethylene (UHMWPE), PEEK, metal and/or porous metal, additionally the joint insert 900 may be prepared with a coating of ceramics, Ultra-high-molecular-weight polyethylene (UHMWPE), PEEK, and/or porous metal. Implants may also be custom made for each patient (i.e. patient specific implants “PSI”) with a combination of manufacturing techniques including additive sintering or 3D printing. The materials for implantation may include porous mesh as well as non-porous fabrication. The implant may include polymer spacers, bone ingrowth portions, or ceramic surfaces.

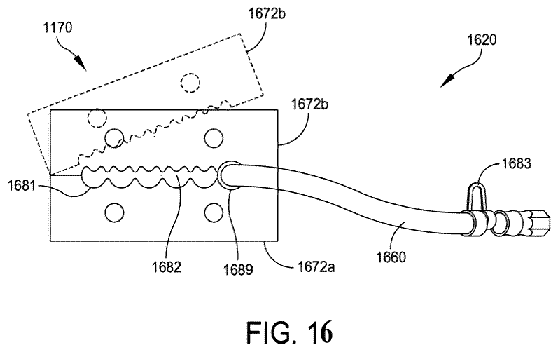

FIG. 16 illustrates a clam shell drill guide according to an embodiment of the disclosed subject matter. As shown in FIG. 16, the drilling assembly 1620 includes a sheath 1660, a counter torque device 1683 and a depth restricting device 1689 preventing the drill bit (not shown) from extending beyond the desired depth. Because of the guide notches 1682 and clearance issues, the drill bit cannot be inserted through the guide holes 1681 in the drill guide 1670. To assist, a clam shell drill guide 1670 may be used, in which the drill guide 1670 is formed of two halves shown as upper half 1672b and the lower half 1672a. In this manner one of the halves may be indexed to the patient's bone in the proper position, the sheath 1660 secured in the lower portion of the guide hole 1681, and the other half 1672b secured to the lower half 1672a, forming a complete guide hole 1681 for guiding the drilling assembly 1620. While a claim shell apparatus is shown for opening and shutting the two halves of the drill guide 1670 in FIG. 16, other mechanisms for connecting the portions is also envisioned, for example screws

FIGS. 12A and 12B illustrate a mating set of joint inserts, an upper insert 1200 and the lower insert 1200b. The upper insert 1200 has a joint surface 1290 proximate the joint and flexibly engages an opposing joint surface 990 of insert 900 as described in FIGS. 9A-9D. The upper insert 1200 likewise has a bone engaging surface 1210 that is shaped as the inverse of the scalloped bone surface intended to engage, for example the tibia. Specifically, the bone engaging surface 1210 includes a plurality of concave ridges 1291 (vs convex ridges 991 for the lower insert 900), each of the concave ridges 1291 defined by a circular cross section 1252 along and arc 1253 as similarly described with respect to insert 900, these concave ridges cooperate with the troughs of the scalloped bone surface defined with substantially similar circular cross sections 252 and arcs 253. Each of the convex ridges 1291 extend along the longitudinal axis and are laterally adjacent to the other concave ridges 1291 across the engaging surface 1210. As the bone engaging surface 1210 of the upper insert 1200 is an inverse of its corresponding scalloped bone surface, the prepared bone surface likewise matching the topography of the bone engaging surface 1210. A peg as discussed with respect to insert 900 may likewise be received into a cooperating peg bore (not shown) in the scalloped bone surface 1210 to aid in rigidly attaching the upper insert 1200 to the bone surface. The peg may be integral to the upper joint insert 1200, or may be inserted subsequently through an aperture (not shown) in the insert 1200.

FIG. 12B illustrates the cooperating joint surfaces of the upper joint insert 1200 and lower joint insert 900 at the cross sections A-A and B-B respectively. As shown in FIG. 12B, the joint surface 1210 of the upper joint insert 1200 substantially matches the lower joint surface 910 of the lower joint insert 900. While not shown, in use the surfaces 1210 and 910 are in contact and transfer loads between the respective bones (e.g. tibia and talus).

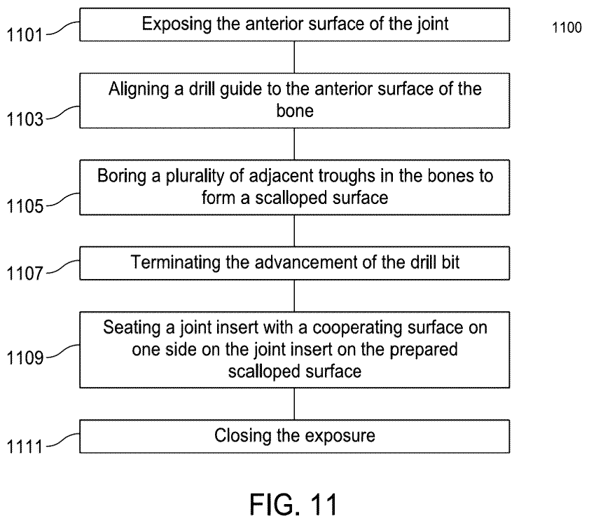

FIG. 11 shows a method 1100 for preparing a scalloped bone surface in the repair of a joint between two bones, specifically an ankle joint. As shown in Block 1101 the patient bone surface is exposed from the anterior surface. A drill guide is then aligned and affixed to the anterior surface of the bone as shown in Block 1103, alternatively the drill guide may be attached to a frame attached to the bone or if a robotic system is used in lieu of the drill guide, it may be registered with respect to the bone surface. Boring a plurality of adjacent troughs in the proximal (or distal) end of one of the bones to form a scalloped surface; by passing a bit attached to an arced sleeve and shaft along multiple predefined arcs into bone as shown in Block 1105, the predefined arcs determine by a drill guide having plural guide bores as discussed above or guided by a robotic controller. Terminating the advancement, or depth, of the drill bit to prevent the plurality of troughs from extending through the posterior rim and sides of the bone as shown in Block 1107. The termination may be accomplished with a measured depth indicator, a stop on the sleeve at a predetermined location preventing the sleeve from extending further through the drill guide, or via robotic control. In some embodiments, the troughs may extend through to the other side of the bone, however, in embodiments where the boring is terminated the unremoved bone beneficially serves to further secure and stabilize the joint insert while the bone heals to the implant 900. Thus, the depth of each bore may be advantageously pre-programed into a computer model or digital surgical plan, based on pre-op or intra-op scans. The desired cut trajectory and depth may be achieved with manual controls, patient specific guides/stops, robotic controllers, or a combination of methods. This depth control is especially useful in total ankle replacement surgery. Additionally, in these procedures plunging a cutting tool too deep, and beyond the posterior cortex of the tibia, is known to risk permanent damage to the posterior neurovascular bundle and other connective tissues. Intentionally not cutting at a depth beyond the posterior cortex will protect sensitive anatomy and stabilize the implant. The joint insert is then seated on the prepared scalloped surface as shown in Block 1109, the joint insert having a cooperating surface that is the inverse of the scalloped surface. The cooperation of the insert surface and the prepared surface stabilize the insert, restricting relative movement between the insert and scalloped surface. The wound created by the exposure is then closed using known methods as shown in Block 1111. To further stabilize the joint insert, a peg hole may also be bored into the scalloped surface and a peg integral to or separated from the insert is positioned in the peg hole. In the embodiment of hemi-arthroplasty, just one bone surface is cut while leaving the other intact. In the case of the ankle, this may include preparation of just the tibia surface while keeping the talus intact. In another embodiment, both bones on either side of the joint may be reamed simultaneously. In the case of total ankle arthroplasty the tibia and talus may be prepared with the arched reaming device.

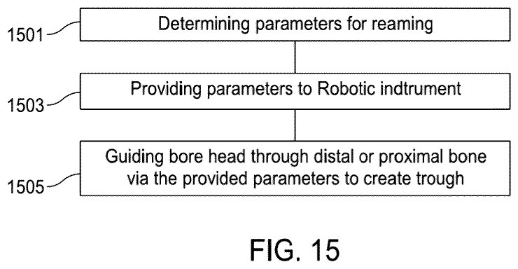

FIG. 15 illustrates an embodiment in which a robotic instrument performs the boring of the troughs in the proximal or distal end of the bone. In this embodiment the parameters of the reaming/boring are established as shown in Block 1501, the parameters may include the size and shape of the troughs, the position with respect to the joint surface, the longitudinal arc of the troughs, the respective angle of the troughs to the bone or adjacent troughs, the bone entry location, the trough terminus location, the rate of boring, and the number of troughs, etc. Once these parameters are defined, they may be provided to the robotic instrument, for example by entering manually or uploading the information as shown in Block 1503. The robotic instrument guided by the parameters may advance the boring head through the proximal or distal end of the bone to form the respective troughs in the bone as shown in Block 1505. It is also envisioned that the robot may be controlled by the surgeon in some respects, for example controlling the advancement speed of the bore head, or making adjustment to the path of the boring head. The degree of real-time control may also be limited by the parameters, or flexible such that the surgeon may engage in some free hand boring. Where surgeon real time control is used, fluoroscopic or virtual feedback would be beneficial in assisting the surgeon in controlling the robotic instrument.

Beneficial applications of the disclosed embodiments and methods are not limited to hemi-arthroplasty, but may also facilitate in addressing bi-polar, hemi, semi, and other osteochondral defects.

While the disclosed subject matter is described with respect to the ankle joint, the use of the systems, methods, guides and implants are likewise envisioned for other joints. Additionally, although the scalloped surface is described herein as being prepared with the use of a drill guide and drill assembly, other preparation techniques implementing computer control and/or robotic machining are also envisioned, with or without utilizing various components of the disclosed systems.

An aspect of the disclosed subject matter is that the general alignment of the troughs in a longitudinal manner improves the ability to position the joint insert (implant) within the joint longitudinally as the maximum thickness of the insert will generally correspond to the maximum opening on the prepared scalloped bone surface, verses lateral insertion or prior art implants, in which the minimum opening on the prepared bone surface would need to match or exceed the maximum thickness of the insert to allow insertion.

Although the methods described above are with reference to the illustrated flowcharts, it will be appreciated that many other ways of performing the acts associated with the methods can be used. For example, the order of some operations may be changed, and some of the operations described may be optional. The foregoing is provided for purposes of illustrating, explaining, and describing embodiments of these disclosures. Modifications and adaptations to these embodiments will be apparent to those skilled in the art and may be made without departing from the scope or spirit of these disclosures.

What is claimed is:

1. A system for repairing a joint, comprising:
a joint insert comprising a scalloped top surface configured to rigidly engage a prepared complimentary bone surface; a bottom surface configured to non-rigidly engage a joint surface, the bottom surface opposing the top surface; and
a boring guide comprising a plurality of bores defined by a cross section and a longitudinal arc, the bores configured to cooperate with a boring assembly having a head that is directed on a predetermined path defined by the longitudinal arc of the respective bores.

2. The system of claim 1, wherein the boring assembly comprises a rigid sheath housing a flexible drive shaft with a bit operatively positioned at a distal end of the rigid sheath so as to be capable of being driven by the flexible drive shaft.

3. The system of claim 1, wherein the longitudinal arc is a constant radius circular arc.

4. The system of claim 1, wherein the longitudinal arc is a constant radius helical arc.

5. The system of claim 1, wherein the bore guide includes a plate having a front face and rear face with one or more bores communicating between the front face and the rear face such that each of the one or more bores is defined by a longitudinal arc and a cross section perpendicular to and along the longitudinal arc.

6. The system of claim 5, wherein the predetermined paths defined by the longitudinal arc define respective non-parallel planes.

7. A joint insert comprising:
a top surface configured to engage a prepared bone surface proximate to a joint and defining a plurality of convex ridges, each convex ridge having an outer surface intersecting the outer surface of laterally adjacent ridges, the outer surface of each ridge defined by a constant radius longitudinal arc and a circular cross section perpendicular to and along the arc;
a bottom surface configured to non-rigidly engage a surface of a bone forming a portion of a joint, wherein the plurality of ridges configure to engage a plurality corresponding concave troughs in the prepared bond surface.

8. The joint insert of claim 7, wherein the joint insert is a patient specific implant.

9. The joint insert of claim 7, wherein the joint insert is 3D printed.

10. The joint insert of claim 7, wherein the joint insert is formed of a material selected from the group consisting of: ceramic, Ultra-high-molecular-weight polyethylene (UHMWPE), PEEK, porous metal.

11. A method for repairing a joint comprising:
forming an incision in a patient so as to expose a joint defined between two bones;
boring a plurality of troughs in at least one of a proximal and a distal end of one of the bones to form a scalloped surface; and,
seating an insert with a cooperating surface on one side on the prepared scalloped surface.

12. The method of claim 11, wherein the step of boring a plurality of adjacent troughs at least one of a proximal and a distal end of one of the bones comprises the steps of:
selecting a drill guide defining a plurality of guide bores, wherein each of the plurality of guide bores are defined by a constant radius longitudinal arc and a circular cross section perpendicular to and along the arc;
positioning the drill guide with respect to the joint; and,
advancing a sleeve of a drill assembly through each of the plurality of guide bores.

13. The method of claim 12, further comprising terminating the advancement of the sleeve to prevent the troughs from extending through an opposite side of the bone.

14. The method of claim 13, wherein the step of terminating the advancement comprises providing a stop on the sleeve at a predetermined location preventing the sleeve from extending further through the drill guide.

15. The method of claim 12, further comprising boring a peg hole through the scalloped surface into the bone.

16. The method of claim 12, wherein the step of boring a plurality of adjacent troughs in the proximal end of one of the bones comprises the steps of:
defining parameters associated with the plurality of adjacent troughs;
providing a robotic boring device with a boring head; and
advancing the boring head through each the proximal or distal end of the bone along a path defined by the parameters.

17. The method of claim 16, wherein each of the plurality of adjacent troughs have a cross section selected from the group consisting of semi-circular shape, semi-elliptical shape, V-shaped, U shaped.

18. The method of claim 16, wherein the boring head is selected from the group consisting of an ultrasonic cutting head, a mechanical bit, and a laser head.

19. The method of claim 16, further comprising terminating the advancement of the bore head to prevent the troughs from extending through an opposite side of the bone.

20. The method of claim 12, further comprising forming the joint insert, wherein the joint insert comprises:
a top surface configured to engage the plurality of adjacent troughs a prepared bone surface proximate to a joint; the top surface having a plurality of convex ridges configure to engage the corresponding plurality of adjacent troughs in the distal or proximate end of the bone; and,
a bottom surface configured to non-rigidly engage a joint surface, the bottom surface opposing the top surface.
